(19) United States
(12) Patent Application Publication
(10) Pub. No.: US 2009/0048570 A1
(43) Pub. Date: Feb. 19, 2009

(54) EXTERNAL URINARY CATHETER

(75) Inventor: Jesper Touborg Jensen, Kokkedal (DK)

Correspondence Address: JACOBSON HOLMAN PLLC, 400 SEVENTH STREET N.W., SUITE 600, WASHINGTON, DC, 20004, US

(73) Assignee: COLOPLAST A/S, Humlebaek (DK)

(21) Appl. No.: 11/661,258

(22) PCT Filed: Aug. 30, 2005
(86) PCT No.: PCT/EP2005/054252

§ 371 Date: Aug. 13, 2008

Related U.S. Application Data
(60) Provisional application No. 60/690,894, filed on Jun. 16, 2005.

(30) Foreign Application Priority Data
Aug. 30, 2004 (DK) PA 2004 01306

(51) Int. Cl. A61F 5/453

(52) U.S. Cl. 604/349

(57) ABSTRACT

An external urinary catheter comprises a sheath portion (1) and a tip portion (2) for receiving a connector. The tip portion (2) is attached to one end of the sheath portion (1) and comprises a seizing area (4) intended for seizing and subsequent guidance of said tip portion (2). The seizing area (4) comprises friction-increasing means (4b), e.g. in the form of ribs or grooves.

The present invention relates to an external urinary catheter with a longitudinal axis, said catheter comprising a sheath portion and a tip portion for receiving a connector, said tip portion being attached to one end of said sheath portion and comprising a seizing area intended for seizing and subsequent guidance of said tip portion.

An external urinary catheter as defined in the introduction is well known in the art and is e.g. described in Applicant's international published applications WO 2004/004796 A1 and WO 91/17728. External urinary catheters are conventionally used in urinary catheter devices for aiding male urinary incontinence and for use in hospitals in connection with treatment and surgery of urethral disorders. Such an external urinary catheter comprises a sheath or body portion enclosing the shaft of the penis, and a tip portion that is provided with a comparatively short discharge tube that via a hose is connected to a urine collection or urinary bag that is e.g. fastened to the bed or the leg of the user. Alternatively, the external urinary catheter, or condom catheter, is arranged externally on the penis and may be a corona urisheath sealing against the foreskin.

The connection of the tip portion and the urinary bag is traditionally obtained by pushing a cone shaped hose connector into a flexible tip connector of the tip portion of the catheter. In order to obtain a safe connection that does not come apart unintentionally, the tip connector and the hose connector have to be pushed together with considerable force. Pulling the connectors apart from each other requires an equally large force.

The hose connector is normally a relatively rigid element that may be easily manipulated by the user. However, the soft, slippery material of the tip portion results in that two strong hands are required in order to connect or release the two parts. Therefore, disabled users most often require help to be able to perform the mounting of the urinary catheter. Such help is costly and since most users consider mounting of urinary catheters to be a private matter, they consider it more or less unpleasant. Furthermore, medical staff or helpers are required to wear gloves when manipulating external urinary catheters. Such gloves are fabricated of a soft and slippery material similar to the material of the urinary catheter and this further aggravates the problem.

As a solution to the described problem several connections based on locking devices have been suggested. However, such devices are bulky, uncomfortable and are not easily manipulated.

Thus, it is the object of the present invention to provide an external urinary catheter of the type mentioned in the introduction, which overcomes or alleviates the described problem.

To meet this object the external urinary catheter is characterized in that said seizing area comprises friction-increasing means.

With such friction-increasing means seizing and manipulating the tip portion is made considerably easier. Also, connecting and releasing of the tip portion and the connector is more readily carried out. Thus, it is possible even for disabled users to handle mounting of the external urinary catheter according to the invention without the need for assistance from a medical staff. As a consequence, users experience a more comfortable mounting and dismounting of the urinary catheter, and medical personnel may be saved.

Moreover, since friction-increasing means may be readily manufactured during the production of the external urinary catheter, the said advantages may be obtained with low or no additional production costs.

In an advantageous embodiment of the invention said friction-increasing means are in the form of ribs or grooves. Ribs and grooves may be manufactured with low expenses and provide for a significantly improved grip of the tip portion. Preferably, said ribs or grooves are in the form of circumferential ribs or grooves extending about said longitudinal axis. This provides friction in the proper direction when connecting or releasing the tip portion and the hose.

Alternatively, said friction-increasing means may be in the form of corrugations or a surface treatment. The surface treatment may be in the form of a coating.

It should be understood that friction-increasing means such as ribs, grooves, corrugations or any other corresponding height modification of the seizing area can generally be referred to as protrusions.

Furthermore, the present invention discloses a method for manufacturing a tip portion for receiving a connector, said tip portion as part of an external urinary catheter comprising a seizing area intended for seizing and subsequent guidance of said tip portion, wherein said seizing area comprises friction-increasing means comprising the step of injection-moulding the tip portion of the catheter into a cavity that is provided with the projections or indentations necessary for producing the friction-increasing means. Injection-moulding advantageously provides a simple, one-step process where the tip portion, usually along with the whole external urinary catheter, is created simultaneously with the friction-increasing means provided thereon. Instead of injection-moulded the tip portion or the whole external urinary catheter can be blow-moulded.

In another embodiment a method is disclosed for manufacturing a tip portion for receiving a connector, said tip portion as part of an external urinary catheter comprising a seizing area intended for seizing and subsequent guidance of said tip portion, wherein said seizing area comprises friction-increasing means comprising the step of surface treating the tip portion of the catheter to produce the friction-increasing means.

Yet another embodiment discloses a method for manufacturing a tip portion for receiving a connector, said tip portion as part of an external urinary catheter comprising a seizing area intended for seizing and subsequent guidance of said tip portion, wherein said seizing area comprises friction-increasing means comprising the step of coating the tip portion of the catheter to produce the friction-increasing means.

The invention will be explained in detail in the following by means of examples of embodiments with reference to the schematic drawing, in which

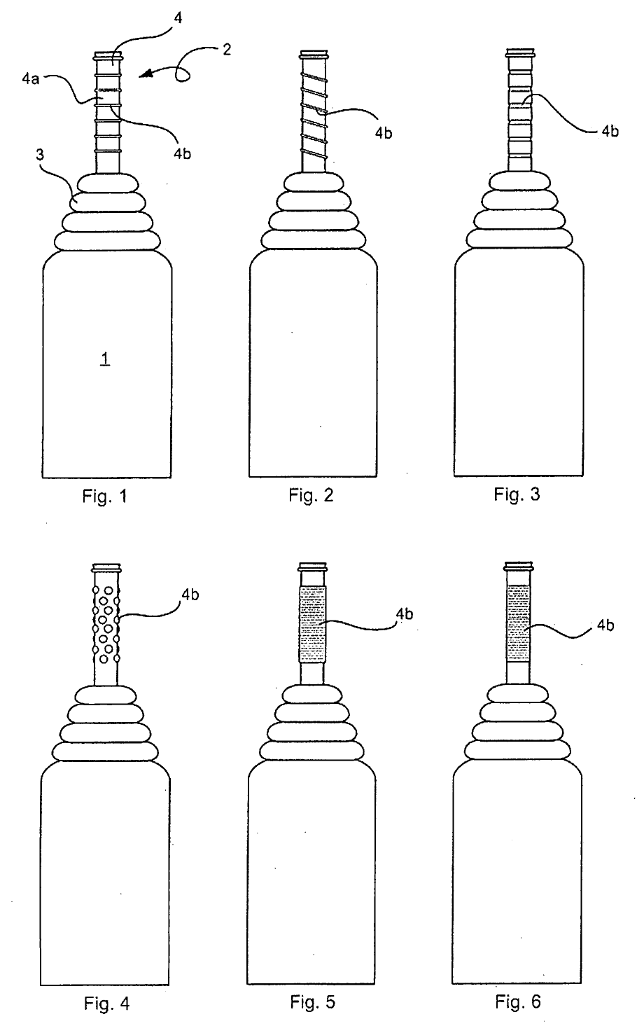

FIG. 1 is a first embodiment of the external urinary catheter according to the invention,

FIG. 2 is a second embodiment of the external urinary catheter according to the invention,

FIG. 3 is a third embodiment of the external urinary catheter according to the invention,

FIG. 4 is a fourth embodiment of the external urinary catheter according to the invention,

FIG. 5 is a fifth embodiment of the external urinary catheter according to the invention, and

FIG. 6 is a sixth embodiment of the external urinary catheter according to the invention

FIG. 1-6 show embodiments of an external urinary catheter according to the invention with a longitudinal axis. Generally, the catheter comprises a circular cylindrical sheath portion 1 for mounting on the penis of a user (not shown) and a tip portion 2 for connecting the urinary catheter to a urinary bag or the like (not shown). The tip portion 2 is attached to the sheath portion 1 at an upper end thereof. In the embodiments shown, the tip portion 2 comprises a flexible bellows portion 3 and a circular cylindrical tip connector 4 for connecting the external urinary catheter to a hose leading to the urinary bag.

The bellows portion 3 comprises four annular parts that may be arbitrarily compressed, whereby the tip connector 4 may be directed at any angle to the longitudinal axis of the external urinary catheter less than approximately 90°. This prevents blockage of the hose leading to the urinary bag and allows for movement of the catheter independently from the urinary bag.

The tip connector 4 comprises a seizing area 4a for seizing by hand and subsequently guiding the tip portion into connection with a hose connector of the hose leading to the urinary bag. The hose connector may e.g. be in the form of a hollow cone, which is pushed into the circular opening of the tip connector 4. In the embodiments shown, the seizing area 4a is the smooth part of the tip connector 4 between the two ends of the tip connector 4. In order to connect the external urinary catheter to the urinary bag, the user seizes the hose connector with one hand and grabs hold of the seizing area 4a of the tip connector 4 with the other hand. Then, the user guides the cone of the hose connector into the tip connector 4 and pushes them into a firm grip with each other. In order to facilitate this seizing and guidance operation, the seizing area 4a is disposed on its surface with friction-increasing means 4b.

In the embodiment of FIG. 1 the friction-increasing means 4b are in the form of six ribs extending circumferentially about and perpendicularly to the longitudinal axis of the external urinary catheter. This disposition of the ribs provides for a large frictional resistance in the direction in which the tip connector 4 is to be pushed during said connecting operation. However, the ribs may also be positioned at an angle to the direction perpendicular to the longitudinal axis of the external urinary catheter, e.g. in a helix shape as shown in FIG. 2. Also, the friction-increasing means may be in an inwardly projecting form, such as grooves as shown in FIG. 3.

The friction-increasing means are not necessarily in the form of ribs or grooves; they may also be in the form of corrugations such as hemisphere-shaped projections or elevations as shown in FIG. 4. The corrugations may also be in the form of indentations and they may be disposed with any suitable shape, size and in any suitable number. They may e.g. be in the form of uniformly distributed circular, oblong, or pyramid-shaped projections or randomly distributed elevations and indentations or they may be a combination of these.

The friction-increasing means may be in the form of a surface treatment. FIG. 5 shows an embodiment of the external urinary catheter according to the invention with friction-increasing means in the form of a coating covering the seizing area 4a. The coating is made from a material with good frictional properties such as rubber. The described advantages may also be reached by means of other surface treatments such as e.g. an abrasive grinding applied directly to the material of the seizing area 4a as shown in FIG. 6.

The external urinary catheter according to the invention may be manufactured in any suitable way and of any suitable material, preferably a plastics material, latex or silicone. The tip portion 4 of the catheter may e.g. be manufactured by injection-moulding it into a cavity that is provided with the projections or indentations necessary for producing any of the friction-increasing means of FIG. 1-4. The other parts of the catheter may be produced in a similar or any other suitable way such as a dipping or extrusion process.

The invention should not be regarded as being limited to the embodiments described in the above but various combinations and modifications may be carried out without departing from the scope of the following claims.

1. An external urinary catheter with a longitudinal axis, said catheter comprising a sheath portion (1) and a tip portion (2) for receiving a connector, said tip portion (2) being attached to one end of said sheath portion (1) and comprising a seizing area (4) intended for seizing and subsequent guidance of said tip portion (2), characterized in that said seizing area (4) comprises friction-increasing means (4b).

2. An external urinary catheter according to claim 1, wherein said friction-increasing means are in the form of protrusions.

3. An external urinary catheter according to claim 2, wherein said friction-increasing means are in the form of ribs or grooves.

4. An external urinary catheter according to claim 3, wherein said ribs or grooves are in the form of circumferential ribs or grooves extending about said longitudinal axis.

5. An external urinary catheter according to claim 1, wherein said friction-increasing means are in the form of corrugations.

6. An external urinary catheter according to claim 1, wherein said friction-increasing means are provided by means of a surface treatment.

7. An external urinary catheter according to claim 6, wherein said surface treatment is in the form of a coating.

8. An external urinary catheter according to claim 1, wherein the catheter and friction-increasing means are mould injected.

9. An external urinary catheter according to claim 8, wherein the catheter and friction-increasing means are mould injected in one piece.

10. An external urinary catheter according to claim 1, wherein the urinary catheter is made of silicone.

11. A method for manufacturing a tip portion (2) for receiving a connector, said tip portion (2) as part of an external urinary catheter comprising a seizing area (4) intended for seizing and subsequent guidance of said tip portion (2), wherein said seizing area (4) comprises friction-increasing means (4b) comprising the step of injection-moulding the tip portion (4) of the catheter into a cavity that is provided with the projections or indentations necessary for producing the friction-increasing means.

12. A method for manufacturing a tip portion (2) for receiving a connector, said tip portion (2) as part of an external urinary catheter comprising a seizing area (4) intended for seizing and subsequent guidance of said tip portion (2), wherein said seizing area (4) comprises friction-increasing means (4b) comprising the step of surface treating the tip portion (4) of the catheter to produce the friction-increasing means.

13. A method for manufacturing a tip portion (2) for receiving a connector, said tip portion (2) as part of an external urinary catheter comprising a seizing area (4) intended for seizing and subsequent guidance of said tip portion (2), wherein said seizing area (4) comprises friction-increasing means (4b) comprising the step of coating the tip portion (4) of the catheter to produce the friction-increasing means.
